(19) United States
(12) Patent Application Publication
(10) Pub. No.: US 2026/0177004 A1
(43) Pub. Date: Jun. 25, 2026

(54) LUBE OIL MONITORING SYSTEM AND METHODS OF USE THEREOF

(71) Applicant: Solar Turbines Incorporated, San Diego, CA (US)

(72) Inventors: David M. Higdon, San Diego, CA (US); Christian M. Cook, Waxhaw, NC (US); Cody W. Allen, Escondido, CA (US); David S. Glaser, Fort Worth, TX (US); Jamaal A. Sanders, La Mesa, CA (US)

(21) Appl. No.: 18/988,526

(22) Filed: Dec. 19, 2024

(51) Int. Cl. F02C 7/14; G01N 33/28

(52) U.S. Cl. CPC F02C 7/14; G01N 33/2888

(57) ABSTRACT

A method and system for detecting degradation of a lube oil temperature control valve in a gas turbine engine. The system includes temperature sensors measuring lube oil tank temperature, heat exchanger outlet temperature, and header temperature downstream of the temperature control valve. Valve position is determined based on temperature measurements and extracted contextual features describing operating conditions. The system determines valve degradation by comparing valve positions to expected positions, analyzing seasonal variations, and detecting deviations from baseline operation. The system predicts potential valve failures before peak ambient temperature periods based on detected degradation rates and historical patterns. Preventative maintenance notifications are generated with recommended timing based on the predicted valve failure timeline.

### TECHNICAL FIELD

The present application is related to monitoring and detecting degradation in temperature control valves used in lube oil cooling systems for gas turbine engines.

### BACKGROUND

Gas turbine engines use lube oil systems to provide cooling and lubrication to bearings and other rotating components. The lube oil system typically includes a tank, pump, heat exchanger, and temperature control valve that regulates oil temperature by mixing cooled and uncooled oil flows. The temperature control valve contains a wax element that expands and contracts with temperature changes to modulate flow between the heat exchanger and bypass paths.

Publication US20190271995A1 discloses a thermostat assembly with a position sensor for controlling coolant fluid flow through an aperture, however, the publication does not describe determining early degradation of passive mechanical valves in a lube oil cooling system. There is therefore a need for methods that can predict early degradation of passive mechanical valves.

### SUMMARY

This document discloses methods, systems, and apparatuses for detecting degradation of temperature control valves in lube oil cooling systems by using temperature measurements to calculate valve position and compare it to expected positions. The system includes temperature sensors at the lube oil tank, heat exchanger outlet, and header locations. A controller processes these temperature measurements to determine the valve's position and monitors for deviation from normal operation patterns that indicate degradation. When degradation is detected, the system generates notifications to enable preventative maintenance before an unexpected shutdown occurs.

In some implementations, a lube oil cooling system for a gas turbine engine includes a lube oil tank, pump, heat exchanger, and temperature control valve. The temperature control valve has dual inlet ports—one receiving oil from the tank and another receiving cooled oil from the heat exchanger—and an outlet port that provides temperature-controlled oil to engine components. Temperature sensors measure various points including tank, heat exchanger outlet, and header temperatures. A controller receives these temperature measurements and uses them to determine the valve's position through calculations. The controller compares this calculated position against expected positions at given temperatures to detect valve degradation. When degradation is detected, indicating the valve is not operating within normal parameters, the system generates a notification. This enables early detection of valve issues before they cause engine shutdowns, particularly during high ambient temperature conditions when proper oil cooling is critical.

In some implementations, a gas turbine engine system includes a lube oil cooling system for temperature management. The cooling system includes a lube oil tank for storage, a pump that circulates oil through the engine system, and a heat exchanger for cooling the oil. A temperature control valve receives both uncooled oil from the tank and cooled oil from the heat exchanger, mixing them to provide temperature-controlled lubrication to the engine's components. Temperature sensors measure at least the tank temperature, while a controller uses these measurements to calculate the valve's position through energy balance equations. The controller compares the calculated position against expected positions at given temperatures to detect valve degradation over time.

In some implementations, degradation is detected in a lube oil temperature control valve using temperature measurements and artificial intelligence (AI). Temperature sensors monitor key points in the system including the lube oil tank, heat exchanger outlet, and header temperatures downstream of the valve. An AI system processes the temperature measurements to determine the valve's actual position using energy balance equations. The system compares this calculated position against expected positions at given temperatures to identify degradation patterns. When degradation is detected, such as the valve opening at progressively higher temperatures compared to its design point, the system can predict potential valve failures.

### BRIEF DESCRIPTION OF THE DRAWINGS

FIG. 1 is a block diagram that illustrates a gas turbine engine system including a lube oil cooling system, in accordance with some aspects of the present technology.

FIG. 2 is a drawing that illustrates an example characteristic curve representing expected positions of a temperature control valve, in accordance with some aspects of the present technology.

FIG. 3 is a flowchart that illustrates an example process for detecting degradation in temperature control valves used in lube oil cooling systems, in accordance with some aspects of the present technology.

FIG. 4 is a flowchart that illustrates an example process for monitoring temperature control valves used in lube oil cooling systems, in accordance with some aspects of the present technology.

FIG. 5 is a flowchart that illustrates an example process for monitoring degradation in temperature control valves, in accordance with some aspects of the present technology.

FIG. 6 is a block diagram that illustrates an example AI system that can implement aspects of the present technology.

FIG. 7 is a block diagram that illustrates an example of a computer system in which at least some operations described herein can be implemented.

### DETAILED DESCRIPTION

Gas turbine engines can use lube oil systems to provide cooling and lubrication to bearings and rotating components. A lube oil system typically includes a tank, pump, heat exchanger, and a temperature control valve, which regulates oil temperature by mixing cooled and uncooled oil flows. Traditional temperature control valves are passive mechanical devices containing a wax element that expands/contracts with temperature changes and a spring that modulates flow between heat exchanger and bypass paths. However, the valves can degrade over time, causing unexpected shutdowns. The present invention provides an innovative solution by using temperature measurements and artificial intelligence (AI) to detect valve degradation before failures occur. The system includes temperature sensors at key points—tank, heat exchanger outlet, and header locations. A controller processes these measurements using energy balance equations to calculate the valve's actual position and compare it to expected positions across operating temperatures. When degradation is detected, such as the valve opening at progressively higher temperatures compared to its design point, the system predicts potential failures. Early warning notifications enable preventative maintenance to be scheduled before high ambient conditions cause shutdowns. The system can also detect heat exchanger degradation by monitoring heat rejection performance when the valve position indicates maximum cooling flow.

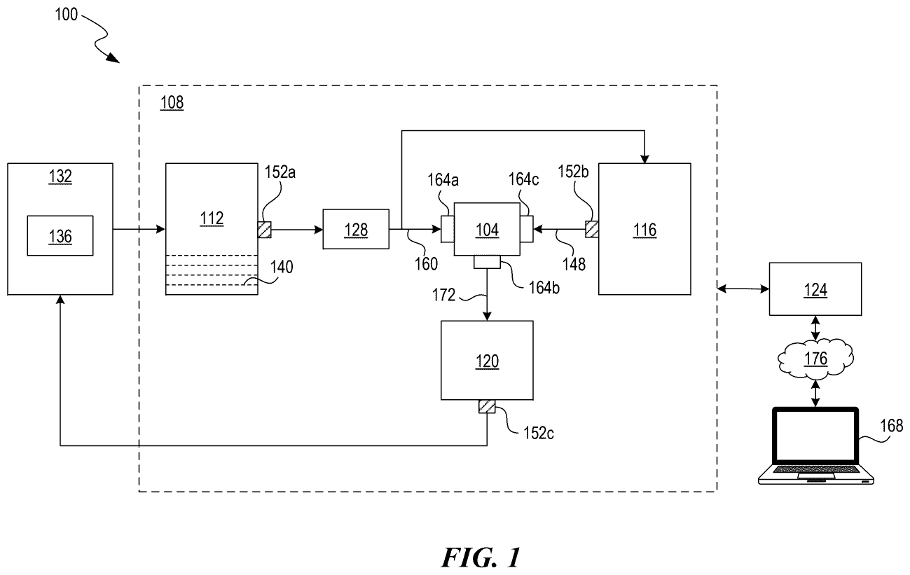

FIG. 1 is a block diagram that illustrates a gas turbine engine system 100 including a lube oil cooling system 108, in accordance with some aspects of the present technology. The lube oil cooling system 108 provides temperature-controlled lubrication to components 136 of the gas turbine engine 132 that require lubrication and cooling during operation. The lube oil cooling system 108 includes a lube oil tank 112 configured to store lube oil 140. A pump 128 is configured to circulate the lube oil 140 from the lube oil tank 112 through the lube oil cooling system 108. In some example implementations, the pump 128 is engine-driven and can operate at approximately 2,030 revolutions per minute (rpm) to provide approximately 140 gallons per minute (gpm) of oil flow when the engine 132 is at normal operating speed.

The gas turbine engine 132 contains several components 136 that can require lubrication and cooling by lube oil. For example, engine bearings support the rotating assemblies of the gas turbine engine 132 and need lubricating oil pumped through them to prevent overheating. A gearbox of the gas turbine engine 132 itself can contain internal components connected to a lube oil pump that require lubrication. Further, driven equipment such as generators or compressors can have bearings that need lubrication and cooling. The lube oil flows through the engine, gearbox, and any rotating machinery that has bearings to maintain proper operating temperatures and prevent component damage.

A heat exchanger 116 is configured to cool the lube oil. The heat exchanger 116 can be either an air-cooled type with fans and cooling fins or a water-cooled plate frame type exchanger. For example, air-cooled heat exchangers can provide approximately 705,000 British Thermal Units per Hour (BTU/hr) of heat rejection capacity, e.g., when cooling oil from approximately 180° F. inlet temperature to approximately 150° F. outlet temperature with approximately 110° F. ambient air.

A temperature control valve 104 is positioned downstream of both the lube oil tank 112 and the heat exchanger 116. The temperature control valve 104 includes multiple ports—specifically a first inlet port 164a configured to receive hot lube oil 160 from the tank 112, a second inlet port 164c configured to receive cooled lube oil 148 from the heat exchanger 116, and an outlet port 164b configured to provide temperature-controlled lube oil 172 to the components 136 of the gas turbine engine 132. The inlet port 164a is used to mechanically couple the heat exchanger 116 to the valve 104. The inlet port 164c is used to mechanically couple the heat exchanger 116 to the valve 104. The outlet port 164b is used to mechanically couple the valve 104 to the header 120.

In some implementations, the temperature control valve 104 has a housing defining internal passages that direct and mix the oil flows from both inlet ports 164ac. The valve housing can incorporate a wax element surrounded by a brass cage with an O-ring and a retaining strap that modulates the internal flow paths. For example, when the lube oil temperature is low, the internal passages are configured to allow flow primarily from inlet port 164a (hot bypass inlet) through to port 164b (common outlet). As temperature increases, the wax element expands, compressing against a spring mechanism, which gradually redirects the internal flow paths to allow more flow from inlet port 164b (cold inlet from the heat exchanger 116) to mix with the hot oil. The internal passages are designed to provide mixing of the hot and cold oil streams in a proportion to achieve the desired outlet temperature at port 164b. The valve housing's internal geometry is configured such that the wax element can modulate between the two flow paths, transitioning from full bypass flow below, e.g., 135° F. to maximum cooler flow by, e.g., 150° F. For example, the temperature range in which the temperature control valve 104 can begin modulating is 100-150° F. In some implementations, the temperature control valve 104 can begin modulating in the 80° F. range.

The ports are typically arranged in a mixing configuration, with inlet port 164c being the cold fluid inlet port receiving oil from the heat exchanger 116 at, e.g., approximately 150° F., and inlet port 164a being the hot bypass fluid inlet receiving uncooled oil from the tank 112 which can reach approximately 180° F. The ports connect to piping that routes the oil flows—the main lube line from the tank 112 connects to inlet port 164a, while the cooler outlet line connects to inlet port 164c. The ports are typically connected using standard pipe fittings compatible with the lube oil system piping specifications. In some implementations, the wax element expands and contracts with temperature changes, working against a spring to modulate the flow split between the hot and cold oil paths. For example, the valve can be designed to begin opening at approximately 100° F. to direct more flow through the heat exchanger 116, becoming fully open by approximately 150° F. to provide maximum cooling. Multiple temperature sensors are positioned at key points in the system 108 to measure temperatures. These include a tank temperature sensor 152a to measure lube oil tank temperature, a heat exchanger outlet temperature sensor 152b to measure cooled oil temperature, and a header temperature sensor 152c positioned downstream of the temperature control valve outlet 164b to measure the final mixed oil temperature.

As oil temperature increases, the wax element softens and expands, pushing against a spring mechanism. The spring's compression modulates the internal flow paths—when cold, the spring keeps the valve 104 positioned to allow primarily bypass flow from the tank inlet port 164a, but as the wax expands with rising temperatures above, e.g., 100° F., it gradually compresses the spring to redirect more flow from the inlet port 164c. This wax-spring interaction continues until the valve 104 reaches full expansion at approximately 150° F., at which point the spring is compressed to maximize flow from the port 164c. The wax element's thermal expansion characteristics can be designed to begin modulation at 100° F. and achieve complete transition by 150° F. to prevent reaching the example 145° F. shutdown temperature. As the wax element degrades over time, it begins expanding at progressively higher temperatures, reducing its ability to properly modulate flow between the ports.

The lube oil cooling system 108 can include pipe fittings to connect the temperature control valve ports 164ac to the header. For example, the inlet port 164a connects to the main lube line from the tank 112 using fittings compatible with the system's piping specifications, allowing hot oil to flow directly from the tank 112 to the valve 104. The second inlet port 164c connects via compatible fittings to the heat exchanger outlet line, enabling cooled oil from the heat exchanger 116 to flow to the valve 104. These mechanical pipe connections can be designed to handle flow rates of approximately 530 L/min (140 gpm) and oil temperatures ranging from 135° F. to 180° F. Other temperature ranges can also be used. The fittings maintain sealing while accommodating the system's operating pressure of up to, e.g., 200 pounds per square inch (PSI) continuous and approximately 250 PSI peak pressure to ensure reliable operation of the temperature control function.

A controller 124 (implemented using digital and/or analog logic) can receive the temperature measurements from the temperature sensors via the header 120. The controller 124 processes these measurements using energy balance equations to determine the actual position of the temperature control valve 104. This calculated position is compared against expected positions at given lube oil temperatures to detect valve degradation over time. The system 100 also includes a remote computer device 168 (e.g., a computer server, a mobile device) connected to the controller through a network/cloud 176. When degradation of the temperature control valve 104 is detected, the controller 124 can generate notifications (sometimes referred to as responses) that are transmitted to the remote device 168. These notifications enable maintenance personnel to schedule preventative maintenance before the degradation leads to an unexpected shutdown.

The temperature control valve position determination is performed using the measured temperatures in an energy balance equation where the header temperature is a weighted average of the tank and cooler outlet temperatures based on the flow split percentage. The flow percentage from the tank versus cooler can be determined using the equation: X=(T_{header}−T_{cooler_outlet})/(Tₜₐₙₖ−T_{cooler_outlet}). Here, X represents the fraction of flow coming from the tank path. This determined position is tracked over time and compared to an expected characteristic curve that represents normal valve operation across the range of operating temperatures. The system 100 is beneficial for preventing unexpected shutdowns during high ambient temperature conditions when proper oil cooling is important. By detecting valve degradation early through position monitoring, maintenance can be scheduled before the valve deteriorates to the point where it fails to provide adequate cooling flow, which would otherwise result in high oil temperatures and a protective shutdown of the engine.

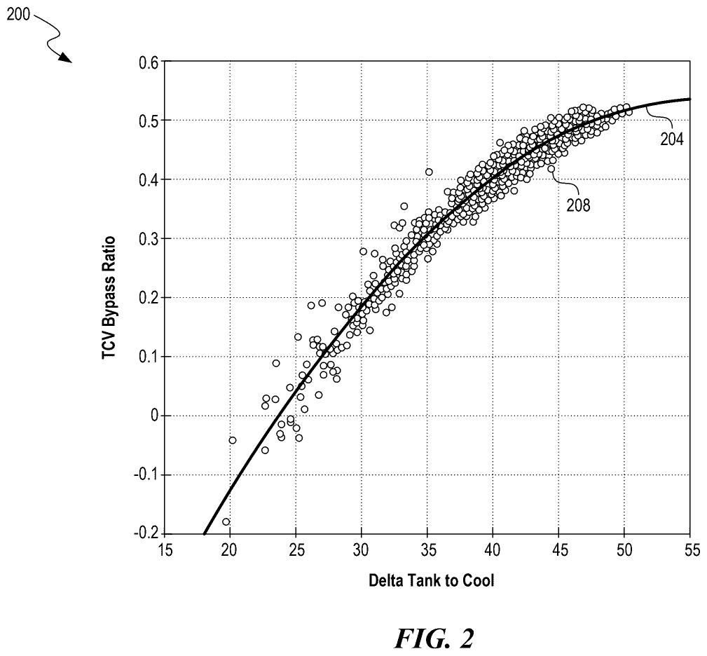

FIG. 2 is a drawing that illustrates an example characteristic curve 200 representing expected positions of a temperature control valve, in accordance with some aspects of the present technology. The characteristic curve 200 plots the temperature control valve bypass ratio (Y-axis) against the temperature difference between the tank and cooler outlets (X-axis). The characteristic curve 200 shows how the temperature control valve modulates flow between the tank and cooler paths as temperatures change. When the temperature difference between tank and cooler is smaller (left side of characteristic curve 200), the bypass ratio is higher, indicating most flow comes from the tank. As the temperature difference increases (moving right on the X-axis), the bypass ratio decreases as the valve directs more flow through the cooler.

The curve 200 includes data points 208 showing actual measured valve positions across different operating conditions. These measured positions can be fit with a quadratic function 204 of the form: TCV(t)=a2ΔT(t)²+a1ΔT(t)+a0, where ΔT(t) is the temperature difference between tank and cooler outlet, and the coefficients ai are determined through least squares regression.

For a properly functioning valve, the characteristic curve 200 shows the valve begins opening at approximately 100° F. to direct flow to the cooler, becoming fully open (bypass ratio near 0) by approximately 150° F. The temperature range in which the temperature control valve 104 can begin modulating is approximately 100-150° F. In some implementations, the temperature control valve 104 can begin modulating in the 80° F. range. The curve provides a baseline for detecting degradation—as the valve wears, its actual position curve will deviate from this expected characteristic (sometimes referred to as a trigger). The system 100 (shown by FIG. 1) monitors for deviations between measured positions and this characteristic curve to identify when a valve is not operating normally. Statistical analysis using confidence intervals around the curve parameters can quantify whether deviations are significant enough to indicate degradation. This enables early detection of valve issues before they cause high temperature shutdowns.

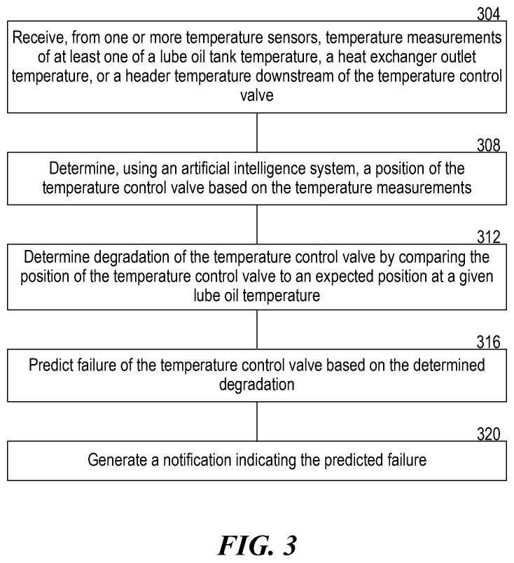

FIG. 3 is a flowchart that illustrates an example process for detecting degradation in temperature control valves used in lube oil cooling systems, in accordance with some aspects of the present technology. In some implementations, the process is performed by the system 100 illustrated and described in more detail with reference to FIG. 1. Particular entities, for example, the controller 124 (shown by FIG. 1) perform some or all of the steps of the process in other implementations. Likewise, implementations can include different and/or additional steps or can perform the steps in different orders.

At 304, a controller receives temperature measurements from temperature sensors positioned at key measurement points in a lube oil cooling system. The sensors can include a tank temperature sensor measuring lube oil tank temperature (T_{TANK_LUBOIL}), a heat exchanger outlet temperature sensor measuring cooled oil temperature (T_{OUTLET_COOLER}), and a header temperature sensor measuring mixed oil temperature downstream of the valve (T_{HDR_LUBOIL}). The sensors can be Class A resistive temperature devices with accuracy of approximately +/−0.3° C. or +/−0.5° F. at approximately 160° F. operating temperature. The temperature measurements can be continuously monitored and collected by the controller at regular intervals, with data sampling rates supporting both 10-minute and 1-hour analysis periods. The controller can receive analog temperature signals from the sensors through standard I/O channels and IRT8/IRT8XT modules. The controller validates the temperature readings by checking that T_{OUTLET_COOLER} is less than or equal to T_{HDR_LUBOIL} which is less than or equal to T_{TANK_LUBOIL} under normal operating conditions. This validation helps identify sensor wiring issues, such as swapped connections, that could affect the valve position calculations.

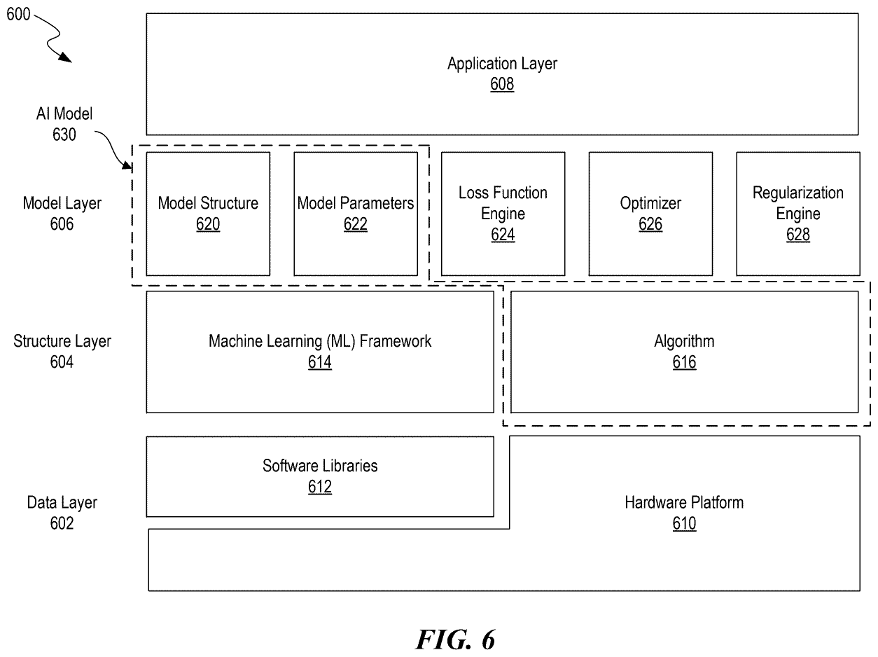

At 308, the controller uses an AI system to determine the temperature control valve position, e.g., by processing temperature measurements through energy balance equations. The AI system used is the same as or similar to the AI system 600 illustrated and described in more detail with reference to FIG. 6. For example, the controller can determine the valve position using position data to make predictions about valve operation. The AI system includes a model layer that implements an AI model using temperature measurement data processed through a structure layer. The model parameters can weight and bias neural network nodes to determine how the temperature input data is transformed into valve position outputs. In implementations, the AI system validates that T_{OUTLET_COOLER}≤T_{HDR_LUBOIL}≤T_{TANK_LUBOIL} before/after performing the position determination.

The controller or a remote computer device can determine a characteristic curve by plotting a valve bypass ratio against the temperature difference between tank and cooler outlet temperatures. The curve follows a quadratic function TCV(t)=a2ΔT(t)²+a1ΔT(t)+a0, where coefficients are determined through least squares regression. The controller can compare the calculated valve position against this curve and its statistical confidence intervals to detect deviations from normal operation.

In some implementations, the controller extracts feature vectors from temperature data including T_{HDR_LUBOIL}, T_{OUTLET_COOLER}, and T_{TANK_LUBOIL} measurements. The AI model can be trained using supervised learning with labeled training data from known good and degraded valve operations. The training data includes temperature patterns, calculated valve positions, and degradation classifications. The model parameters are improved during training using techniques like gradient descent to minimize the loss function and improve prediction accuracy. The system continuously retrains the model as new operational data and degradation patterns are detected.

At 312, the controller determines valve degradation, e.g., by comparing the determined position against a characteristic curve representing expected valve operation. The curve can follow the quadratic function described above, where ΔT is the temperature difference between tank and cooler outlet. For normal operation, the valve can begin opening at 135° F. and becomes fully open by 150° F. The controller monitors for deviations from this expected behavior by comparing the actual position against statistical confidence intervals around the curve parameters. When the valve starts opening at progressively higher temperatures compared to the 135° F. design point (sometimes referred to as a trigger), this indicates degradation. The controller tracks both the absolute temperature deviation from the expected 100° F. opening point as well as the rate of change in opening temperature over time to quantify degradation. The temperature range in which the temperature control valve 104 can begin modulating is approximately 100-150° F. In some implementations, the temperature control valve 104 can begin modulating in the 80° F. range. Significant deviations outside the 90% confidence intervals of the characteristic curve parameters indicate abnormal valve operation requiring maintenance.

The controller can monitor valve degradation through two methods. First, the controller can track the temperature at which flow begins diverting to the cooler by analyzing the valve position by (T_{HDR_LUBOIL}−T_{OUTLET_COOLER})/(T_{TANK_LUBOIL}−T_{OUTLET_COOLER}). Second, the controller can specifically monitor when the opening temperature increases above the exemplary 100° F. design point. The controller can use regression analysis to track both the absolute temperature deviation from 135° F. and the rate of temperature increase over time. When the opening temperature rises toward, e.g., 140° F., this indicates significant degradation requiring maintenance before reaching the 145° F. shutdown threshold.

In some implementations, the controller determines valve degradation by analyzing the temperature differentials between key measurement points. The controller can determine ΔT between tank and cooler outlet (T_{TANK_LUBOIL}−T_{OUTLET_COOLER}), header and cooler outlet (T_{HDR_LUBOIL}−T_{OUTLET_COOLER}), and tank and header (T_{TANK_LUBOIL}−T_{HDR_LUBOIL}). Under normal operation, T_{OUTLET_COOLER}≤T_{HDR_LUBOIL}≤T_{TANK_LUBOIL}, with approximately 30° F. differential between tank and cooler temperatures when the valve is fully open. Deviations from these expected temperature relationships indicate valve degradation. The system validates that the temperature differentials follow the expected pattern to ensure sensor readings are valid before performing degradation analysis.

At 316, the controller predicts valve failure by analyzing the rate of change in valve opening temperature over time. When degradation is detected, such as the valve opening at progressively higher temperatures compared to its design point, the controller determines a predicted time to failure based on the degradation rate. An AI model 630 can process historical temperature data to establish baseline operation patterns and use regression techniques to forecast when the valve will reach critical degradation thresholds. For example, if the valve opening temperature has increased from 100° F. to 138-140° F., and continues rising at a consistent rate, the AI system predicts potential failure before reaching the 145° F. shutdown temperature. The prediction can consider seasonal temperature patterns to estimate when high ambient conditions will make the degraded valve performance critical. This enables maintenance to be scheduled before peak temperature periods when cooling issues are most likely to cause shutdowns.

At 320, the controller generates notifications, e.g., through a remote monitoring diagnostic application that creates alerts and detailed reports (sometimes referred to as responses). When valve degradation is detected, the controller can send notifications to remote computer devices through a cloud network connection. The notifications can include specific maintenance recommendations and timing guidance based on seasonal temperature patterns. For example, if the valve opening temperature has increased from 100° F. toward 140° F., the system generates an alert recommending service before peak summer temperatures. Notifications can include plots showing the valve's operating curve compared to normal baseline operation, temperature trend data, and a predicted timeline to failure. Alerts can be classified into categories including “Temperature Control Valve Service Recommendation”, “Bad Heat Rejection”, and “Degradation Over Time” to help maintenance teams identify the specific issue requiring attention. The notifications enable preventative maintenance to be scheduled before the degradation leads to an unexpected shutdown.

In some implementations, the controller determines heat exchanger degradation by calculating heat rejection when the valve position indicates maximum cooling flow. For a properly functioning heat exchanger, the heat rejection should be approximately 705,000 BTU/hr when cooling oil from approximately 180° F. to approximately 150° F. with approximately 110° F. ambient air. The controller can compare the actual temperature drop across the heat exchanger against this expected 30° F. design point when the valve is fully open. When the temperature drop decreases below expected values despite maximum cooling flow (X approaching 0), this indicates degradation of heat exchanger performance requiring cleaning or maintenance.

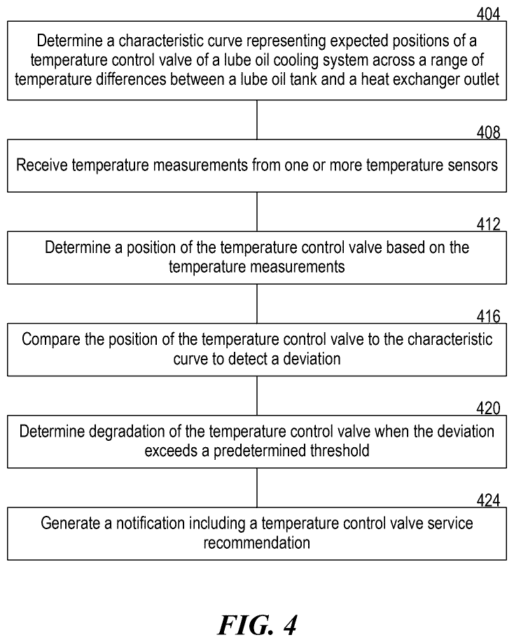

FIG. 4 is a flowchart that illustrates an example process for monitoring temperature control valves used in lube oil cooling systems, in accordance with some aspects of the present technology. In some implementations, the process is performed by the system 100 illustrated and described in more detail with reference to FIG. 1. In some implementations, the process is performed by a lube oil cooling system for a gas turbine engine that includes a tank for storing oil, a pump that circulates oil through the system, and a heat exchanger for cooling the oil. The temperature control valve contains dual inlet ports - one receiving oil directly from the tank and another receiving cooled oil from the heat exchanger. The valve's outlet port provides temperature-controlled oil to engine components such as bearings and rotating components. Temperature sensors measure key points including tank temperature, heat exchanger outlet temperature, and header temperature downstream of the valve. The system maintains proper oil temperatures for bearing lubrication and cooling while enabling monitoring of valve position and performance through temperature measurements. Particular entities, for example, the controller 124 (shown by FIG. 1) perform some or all of the steps of the process in other implementations. Likewise, implementations can include different and/or additional steps or can perform the steps in different orders.

At 404, a controller determines a characteristic curve by plotting a determined valve position against the temperature difference ΔT between tank and cooler outlet temperatures. The curve follows a quadratic function where the coefficients are determined through least squares regression of historical operating data. For normal operation, the curve shows the valve beginning to open at 100° F. tank temperature and becoming fully open by 150° F., with the bypass ratio decreasing from 1.0 to 0.0 across this range. The temperature range in which the temperature control valve 104 can begin modulating is approximately 100-150° F. In some implementations, the temperature control valve 104 can begin modulating in the 80° F. range. The system establishes statistical confidence intervals around the curve parameters to define the expected operating envelope.

At 408, the controller receives temperature measurements from sensors positioned at key points in the lube oil cooling system—including the tank temperature (T_{TANK_LUBOIL}), heat exchanger outlet temperature (T_{OUTLET_COOLER}), and header temperature (T_{HDR_LUBOIL}) downstream of the temperature control valve. The temperature sensors are configured to provide continuous temperature data to the controller through standard resistive temperature devices (RTDs) with Class A accuracy of approximately +/−0.5° F. at 160° F. The controller processes these temperature measurements in real-time to enable monitoring of the lube oil system operation.

At 412, the controller determines a bypass flow percentage X representing the ratio of tank flow versus cooler flow. The controller monitors this percentage over time, comparing it to expected values where X should decrease from 100% to 0% as tank temperature rises from 100° F. to 150° F. Degradation is detected when the bypass percentage deviates from the expected quadratic characteristic curve or when the flow ratios don't match the expected temperature-based operating envelope. For example, the controller determines the valve position by calculating a bypass ratio using the temperature measurements from the sensors. This ratio represents the percentage of flow coming from the tank versus the cooler, with X=1.0 indicating 100% tank flow and X=0.0 indicating 100% cooler flow. The calculation assumes adiabatic flow through the valve and constant specific heat of the oil. The controller validates that T_{OUTLET_COOLER}≤T_{HDR_LUBOIL}≤T_{TANK_LUBOIL} and can use at least a 10° F. differential between tank and cooler temperatures for more accurate position calculation.

At 416, the controller compares the calculated valve position against the characteristic curve by analyzing both the absolute deviation from expected values and statistical confidence intervals. For normal operation, the bypass ratio X should decrease from 1.0 to 0.0 as tank temperature rises from 100° F. to 150° F. The system detects degradation when the calculated position deviates from the expected quadratic curve TCV(t)=a2ΔT(t)²+a1ΔT(t)+a0 by more than the established 90% confidence intervals around the curve parameters (sometimes referred to as a trigger). Additionally, when the valve opening temperature increases above 135° F. or the rate of position change differs significantly from the expected curve, this indicates degradation requiring maintenance.

At 420, the controller determines valve degradation by determining the valve position and comparing it to expected values. For normal operation, the valve begins opening at 100° F. tank temperature and becomes fully open by around 150° F. The system detects degradation when the valve opens at progressively higher temperatures compared to the 100° F. design point or when the calculated position deviates significantly from the expected quadratic characteristic curve TCV(t).

At 424, the controller generates notifications, e.g., through a remote monitoring platform when valve degradation is detected. The notifications can include detailed reports (sometimes referred to as responses) showing temperature measurements, valve position calculations, and specific maintenance recommendations. For valve failures, the controller can recommend inspecting the valve for leaks and correct bypass operation, checking temperature control settings, and verifying oil quality meets specifications. The notifications can also include plots showing the valve's operating curve compared to expected behavior and highlight when the opening temperature has increased above the 100° F. design point.

In some implementations, the controller generates alerts for cooling system component failures by analyzing temperature patterns and system behavior. For air-cooled systems, the controller can detect fan and belt failures by monitoring sudden temperature increases, e.g., seeing header temperature rise by 13-40° F. within 7-22 minutes when a fan or belt fails. For water-cooled systems, coolant pump failures can be identified through rapid temperature increases and reduced heat rejection performance. The system can validate whether the temperature control valve is functioning properly by checking its position calculation before attributing issues to the cooling components. When component failures are detected, the controller can generate detailed notifications, e.g., through an INSIGHT platform specifying the failed component and including maintenance recommendations like checking for loose/worn drive belts, verifying fan blade positions and airflow, or ensuring sufficient water flow through the heat exchanger. The alerts can include plots showing the sudden temperature changes that indicate component failure versus normal operation.

In some implementations, the controller monitors the temperature at which the valve begins opening by analyzing the calculated valve position over time. Under normal operation, the valve begins opening at 100° F. tank temperature, so the system tracks deviations from this baseline. The controller determines the rate of change by calculating how quickly the opening temperature increases from the 100° F. design point toward the 145° F. shutdown threshold. For example, if the opening temperature moves from 100° F. to 138° F. to 140° F. over time, the system calculates this progression rate. Using regression analysis of the temperature trend data, the controller can predict the timeframe when the valve will reach critical degradation requiring maintenance. The predictions account for seasonal temperature patterns to prevent shutdowns during peak ambient conditions. When the rate of temperature increase indicates the valve will soon reach the example 145° F. shutdown threshold, the controller generates preventative maintenance notifications.

The controller can generate heat exchanger degradation alerts by monitoring heat rejection performance, e.g., when the temperature control valve position indicates maximum cooling flow. The controller can determine heat rejection by analyzing the temperature differential between tank and cooler outlet temperatures, with normal operation expecting at least a 30° F. temperature drop across the heat exchanger. When the temperature differential falls below expected values despite maximum cooler flow, the controller identifies degradation requiring cleaning. For air-cooled systems, alerts can recommend checking for blockages in heat exchanger air passageways and cleaning the cooling core per original equipment manufacturer (OEM) specifications. For water-cooled systems, notifications can indicate potential oil fouling inside the cooler or insufficient water flow. The controller can validate valve operation before attributing reduced cooling performance to heat exchanger degradation. Alerts can include plots showing degraded heat rejection performance compared to normal operation baselines.

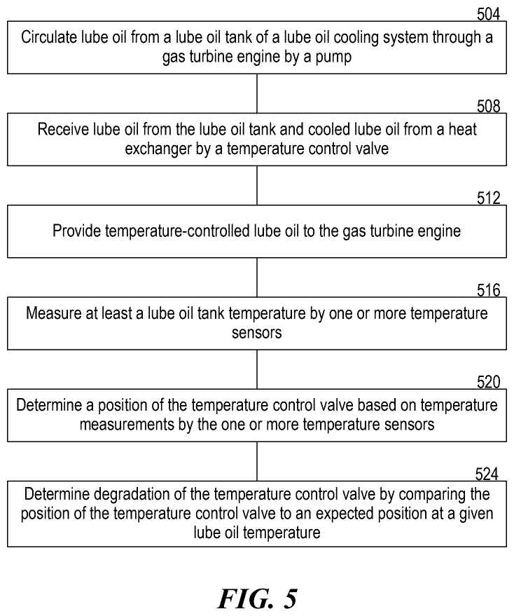

FIG. 5 is a flowchart that illustrates an example process for monitoring degradation in temperature control valves, in accordance with some aspects of the present technology. In some implementations, the process is performed by the system 100 illustrated and described in more detail with reference to FIG. 1. In some implementations, the process is performed by a gas turbine engine system that includes a gas turbine engine with bearings and rotating components and a lube oil system that provides cooling and lubrication by circulating oil through a tank mounted on the engine package skid. For example, the tank stores lube oil that meets Solar specification ES9-224, with typical options including C32/C46 Petroleum oil with specific gravity of 0.8375 at 150° F., or other approved oils like MIL-L-23699. The system can operate with oil temperatures ranging from approximately −18° C. to approximately +107° C. (0° F. to +225° F.) and can handle altitudes up to 8000 ft. The lube oil tank is integrated into the package alongside other key components such as the pump, filters, and heat exchanger to maintain proper oil temperature and flow to the engine's components. Likewise, implementations can include different and/or additional steps or can perform the steps in different orders.

At 504, an engine-driven main lube oil pump circulates oil from the tank through the gas turbine engine system, e.g., at approximately 530 Liters pe minute (L/min) or approximately 140 gpm when operating at its design speed of approximately 2,030 rpm. The pump can be driven by a mechanical connection to the engine's reduction gearbox and can use approximately 17.9 kilowatt (kW) or 24 horsepower (HP) of input power at approximately 200 PSI. The pump circulates oil through multiple paths—directing flow through the heat exchanger for cooling, through a bypass line controlled by the temperature control valve, and through a pressure relief valve that recycles excess flow back to the tank. The system maintains proper oil flow to provide lubrication and cooling to bearings and other rotating components while operating within pressure ranges of approximately 16.88 kilopascal (kPa) minimum inlet to approximately 1380 kPa continuous output pressure. The lube oil cooling system maintains header temperature below approximately 145° F. shutdown threshold by circulating temperature-controlled oil through the engine bearings and rotating components. The temperature control valve regulates oil temperature by mixing cooled oil from the heat exchanger (at approximately 150° F.) with tank oil, beginning modulation at approximately 100° F. and becoming fully open to cooler flow by approximately 150° F. The temperature range in which the temperature control valve 104 can begin modulating is approximately 100-150° F. In some implementations, the temperature control valve 104 can begin modulating in the 80° F. range.

At 508, a heat exchanger cools the lube oil using either an air-cooled or water-cooled configuration. For air-cooled systems, the heat exchanger functions like a radiator with a fan providing forced draft cooling, e.g., capable of rejecting approximately 705,000 BTU/hr of heat with air flow of approximately 28,629 standard cubic feet per minute (SCFM) at design conditions. A temperature control valve contains a wax element that expands/contracts with temperature changes and a spring that modulates flow between heat exchanger and bypass paths. The valve can have dual inlet ports, e.g., port 164c receives cooled oil from the heat exchanger at approximately 150° F. while port 164a receives uncooled oil directly from the tank which can be up to approximately 180° F. The valve mixes these flows to maintain proper oil temperature, beginning to open at 100° F. tank temperature and becoming fully open to the cooler by 150° F. For water-cooled systems, a plate frame heat exchanger design is used instead of the air-cooled radiator configuration.

At 512, temperature-controlled lube oil is provided to the components of the gas turbine engine through a header pipe system downstream of the temperature control valve. The valve mixes hot oil from the tank with cooled oil from the heat exchanger to maintain proper operating temperatures between approximately 18° C. to approximately +107° C. (0° F. to +225° F.). The mixed oil flows through filters before entering the header, which distributes the temperature-controlled oil to the engine bearings, gearbox, generator, and compressor bearings to provide both lubrication and heat rejection from the bearing materials. For example, valve degradation can be determined from temperature differentials between key measurement points in the system. The controller monitors when these differentials deviate from expected patterns, particularly when the difference between header and cooler outlet temperatures indicates the valve is not opening at its design point. By comparing the measured temperature differentials against statistical confidence intervals around normal operating ranges, the system can detect when the valve begins operating outside expected parameters (sometimes referred to as a trigger).

At 516, the system uses temperature sensors placed to measure the lube oil tank temperature, which can range from approximately −18° C. to approximately +107° C. (0° F. to +225° F.) during normal operation. The tank temperature measurement represents the hot oil temperature before cooling and helps determine when the temperature control valve should begin opening. The system can validate sensor accuracy by checking for illogical temperature readings, such as when tank temperature appears lower than cooler outlet temperature, which could indicate incorrect sensor wiring.

At 520, the controller determines valve position by using temperature measurements. The determination provides the valve's actual position without requiring a physical position sensor. The controller processes measurements from temperature sensors at the tank, heat exchanger outlet, and header locations to continuously monitor valve position through these energy balance equations. For example, valve opening temperature is monitored by analyzing temperature measurements from sensors at the tank, heat exchanger outlet, and header locations. The controller detects when the valve begins diverting flow at temperatures higher than its design point. Using the valve position, the controller identifies when the opening temperature exceeds predetermined thresholds, as the valve should begin modulating at 100° F. and be fully open by around 150° F. The system generates alerts when the opening temperature approaches an example shutdown threshold of 145° F.

At 524, the controller determines valve degradation by comparing the calculated valve position against an expected characteristic curve that represents normal operation. The valve should begin opening at approximately 100° F. and be fully open by 150° F.—a deviation from this pattern can indicate degradation. The system monitors when the valve starts opening at progressively higher temperatures compared to its exemplary design point, such as opening at 138° F. or 140° F. instead of 100° F. The controller uses statistical analysis to establish confidence intervals around the expected valve position curve, allowing detection of abnormal operation when the actual position falls outside these boundaries. This enables early detection of degradation before the valve reaches the example shutdown temperature of 145° F. For example, baseline operation is established by constructing a characteristic curve representing expected valve positions across operating temperatures. Deviations from this baseline curve are monitored using statistical analysis with confidence intervals to detect abnormal operation. When the valve begins opening at progressively higher temperatures compared to baseline, failure is predicted before reaching the 145° F. shutdown temperature by analyzing the rate of degradation. The system validates predictions by comparing actual valve positions against the expected characteristic curve and statistical boundaries.

In some implementations, the AI system described with reference to FIG. 6 analyzes historical temperature data stored in the system history to identify seasonal patterns and their impact on valve operation. For example, temperature measurements are processed using machine learning models implemented through layers including data, structure, model and application layers to detect degradation patterns. Based on this analysis, the controller generates notifications when the valve opening temperature increases. The notifications can include maintenance timing recommendations considering seasonal temperature patterns, predicted failure timelines based on degradation rates, and/or detailed reports (sometimes referred to as responses) showing how valve operation has deviated from normal parameters. This enables preventative maintenance to be scheduled before high ambient temperature periods.

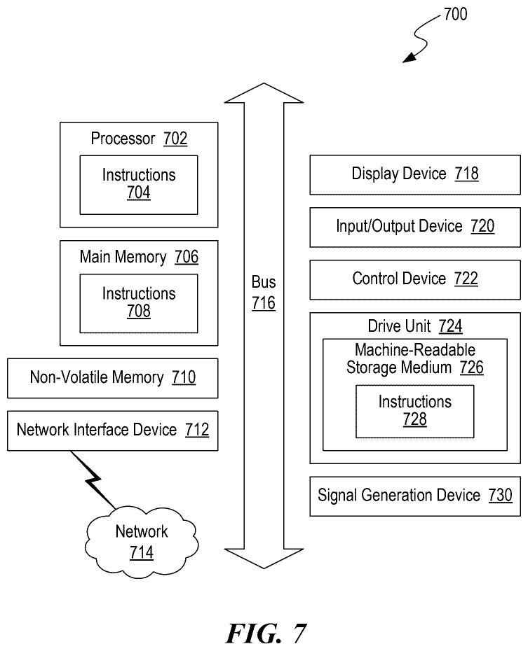

FIG. 6 is a block diagram that illustrates an example AI system 600 that can implement aspects of the present technology. The AI system 600 is implemented using components of the example computer system 700 illustrated and described in more detail with reference to FIG. 7. For example, the AI system 600 can be implemented on the processor 702 using instructions 708 programmed in the memory 706 illustrated and described in more detail with reference to FIG. 7. Likewise, implementations of the AI system 600 can include different and/or additional components or be connected in different ways.

FIG. 6 illustrates a layered architecture of AI system 600 that can implement AI models within the controller 124 and/or computer device 168 of FIG. 1, in accordance with some implementations of the present technology. As shown, the AI system 600 can include a set of layers, which conceptually organize elements within an example network topology for the AI system's architecture to implement a particular AI model 630. Generally, an AI model 630 is a computer-executable program implemented by the AI system 600 that analyses data to make predictions. Information can pass through each layer of the AI system 600 to generate outputs for the AI model 630. The layers can include a data layer 602, a structure layer 604, a model layer 606, and an application layer 608. The algorithm 616 of the structure layer 604 and the model structure 620 and model parameters 622 of the model layer 606 together form an example AI model 630. The optimizer 626, loss function engine 624, and regularization engine 628 work to refine and optimize the AI model 630, and the data layer 602 provides resources and support for application of the AI model 630 by the application layer 608.

The data layer 602 acts as the foundation of the AI system 600 by preparing data for the AI model 630. As shown, the data layer 602 can include two sub-layers: a hardware platform 610 (e.g., the controller 124 and/or computer device 168 described in more detail with reference to FIG. 1) and one or more software libraries 612. The hardware platform 610 can be designed to perform operations for the AI model 630 and include computing resources for storage, memory, logic and networking, such as the resources described in relation to FIG. 7. The hardware platform 610 can process amounts of data using one or more servers. The servers can perform backend operations such as matrix calculations, parallel calculations, machine learning (ML) training, and the like. Examples of servers used by the hardware platform 610 include central processing units (CPUs) and graphics processing units (GPUs). CPUs are electronic circuitry designed to execute instructions for computer programs, such as arithmetic, logic, controlling, and input/output (I/O) operations, and can be implemented on integrated circuit (IC) microprocessors. GPUs are electric circuits that were originally designed for graphics manipulation and output but may be used for AI applications due to their vast computing and memory resources. GPUs use a parallel structure that generally makes their processing more efficient than that of CPUs. In some instances, the hardware platform 610 can include computing resources, (e.g., servers, memory, etc.) offered by a cloud services provider. The hardware platform 610 can also include computer memory for storing data about the AI model 630, application of the AI model 630, and training data for the AI model 630. The computer memory can be a form of random-access memory (RAM), such as dynamic RAM, static RAM, and non-volatile RAM.

The software libraries 612 can be thought of suites of data and programming code, including executables, used to control the computing resources of the hardware platform 610. The programming code can include low-level primitives (e.g., fundamental language elements) that form the foundation of one or more low-level programming languages, such that servers of the hardware platform 610 can use the low-level primitives to carry out specific operations. The low-level programming languages do not require much, if any, abstraction from a computing resource's instruction set architecture, allowing them to run quickly with a small memory footprint. Examples of software libraries 612 that can be included in the AI system 600 include INTEL Math Kernel Library, NVIDIA cuDNN, EIGEN, and OpenBLAS.

The structure layer 604 can include an ML framework 614 and an algorithm 616. The ML framework 614 can be thought of as an interface, library, or tool that allows users to build and deploy the AI model 630. The ML framework 614 can include an open-source library, an application programming interface (API), a gradient-boosting library, an ensemble method, and/or a deep learning toolkit that work with the layers of the AI system facilitate development of the AI model 630. For example, the ML framework 614 can distribute processes for application or training of the AI model 630 across multiple resources in the hardware platform 610. The ML framework 614 can also include a set of pre-built components that have the functionality to implement and train the AI model 630 and allow users to use pre-built functions and classes to construct and train the AI model 630. Thus, the ML framework 614 can be used to facilitate data engineering, development, hyperparameter tuning, testing, and training for the AI model 630. Examples of ML frameworks 614 that can be used in the AI system 600 include TENSORFLOW, PYTORCH, SCIKIT-LEARN, KERAS, LightGBM, RANDOM FOREST, and AMAZON WEB SERVICES.

The algorithm 616 can be an organized set of computer-executable operations used to generate output data from a set of input data and can be described using pseudocode. The algorithm 616 can include complex code that allows the computing resources to learn from new input data (e.g., temperature measurements and valve positions) and create new/modified outputs based on what was learned. In some implementations, the algorithm 616 can build the AI model 630 through being trained while running computing resources of the hardware platform 610. This training allows the algorithm 616 to make predictions or decisions without being explicitly programmed to do so. Once trained, the algorithm 616 can run at the computing resources as part of the AI model 630 to make predictions or decisions, improve computing resource performance, or perform tasks. The algorithm 616 can be trained using supervised learning, unsupervised learning, semi-supervised learning, and/or reinforcement learning.

Using supervised learning, the algorithm 616 can be trained to learn patterns (e.g., map input data to output data) based on labeled training data. The training data may be labeled by an external user or operator. For instance, a user may collect a set of training data, such as by capturing data from sensors, images from a camera, outputs from a model, and the like. In an example implementation, training data can include native-format data collected (e.g., in the form of temperature measurements or valve positions) from various source computing systems described in relation to FIG. 1. Furthermore, training data can include pre-processed data generated by various sensors of the system 100 described in relation to FIG. 1. The user may label the training data based on one or more classes and trains the AI model 630 by inputting the training data to the algorithm 616. The algorithm determines how to label the new data based on the labeled training data. The user can facilitate collection, labeling, and/or input via the ML framework 614. In some instances, the user may convert the training data to a set of feature vectors for input to the algorithm 616. Once trained, the user can test the algorithm 616 on new data to determine if the algorithm 616 is predicting accurate labels for the new data. For example, the user can use cross-validation methods to test the accuracy of the algorithm 616 and retrain the algorithm 616 on new training data if the results of the cross-validation are below an accuracy threshold.

Supervised learning can involve classification and/or regression. Classification techniques involve teaching the algorithm 616 to identify a category of new observations based on training data and are used when input data for the algorithm 616 is discrete. Said differently, when learning through classification techniques, the algorithm 616 receives training data labeled with categories (e.g., classes) and determines how features observed in the training data (e.g., valve positions) relate to the categories. Once trained, the algorithm 616 can categorize new data by analyzing the new data for features that map to the categories. Examples of classification techniques include boosting, decision tree learning, genetic programming, learning vector quantization, k-nearest neighbor (k-NN) algorithm, and statistical classification.

Regression techniques involve estimating relationships between independent and dependent variables and are used when input data to the algorithm 616 is continuous. Regression techniques can be used to train the algorithm 616 to predict or forecast relationships between variables. To train the algorithm 616 using regression techniques, a user can select a regression method for estimating the parameters of the model. The user collects and labels training data that is input to the algorithm 616 such that the algorithm 616 is trained to understand the relationship between data features and the dependent variable(s). Once trained, the algorithm 616 can predict missing historic data or future outcomes based on input data. Examples of regression methods include linear regression, multiple linear regression, logistic regression, regression tree analysis, least squares method, and gradient descent. In an example implementation, regression techniques can be used, for example, to estimate and fill-in missing data for machine-learning based pre-processing operations.

Under unsupervised learning, the algorithm 616 learns patterns from unlabeled training data. In particular, the algorithm 616 is trained to learn hidden patterns and insights of input data, which can be used for data exploration or for generating new data. Here, the algorithm 616 does not have a predefined output, unlike the labels output when the algorithm 616 is trained using supervised learning. Said another way, unsupervised learning is used to train the algorithm 616 to find an underlying structure of a set of data, group the data according to similarities, and represent that set of data in a compressed format.

A few techniques can be used in supervised learning: clustering, anomaly detection, and techniques for learning latent variable models. Clustering techniques involve grouping data into different clusters that include similar data, such that other clusters contain dissimilar data. For example, during clustering, data with possible similarities remain in a group that has less or no similarities to another group. Examples of clustering techniques density-based methods, hierarchical based methods, partitioning methods, and grid-based methods. In one example, the algorithm 616 may be trained to be a k-means clustering algorithm, which partitions _n_ observations in _k_ clusters such that each observation belongs to the cluster with the nearest mean serving as a prototype of the cluster. Anomaly detection techniques are used to detect previously unseen rare objects or events represented in data without prior knowledge of these objects or events. Anomalies can include data that occur rarely in a set, a deviation from other observations, outliers that are inconsistent with the rest of the data, patterns that do not conform to well-defined normal behavior, and the like. When using anomaly detection techniques, the algorithm 616 may be trained to be an Isolation Forest, local outlier factor (LOF) algorithm, or K-nearest neighbor (k-NN) algorithm. Latent variable techniques involve relating observable variables to a set of latent variables. These techniques assume that the observable variables are the result of training on the latent variables and that the observable variables have nothing in common after controlling for the latent variables. Examples of latent variable techniques that may be used by the algorithm 616 include factor analysis, item response theory, latent profile analysis, and latent class analysis.

The model layer 606 implements the AI model 630 using data from the data layer and the algorithm 616 and ML framework 614 from the structure layer 604, thus enabling decision-making capabilities of the AI system 600. The model layer 606 includes a model structure 620, model parameters 622, a loss function engine 624, an optimizer 626, and a regularization engine 628.

The model structure 620 describes the architecture of the AI model 630 of the AI system 600. The model structure 620 defines the complexity of the pattern/relationship that the AI model 630 expresses. Examples of structures that can be used as the model structure 620 include decision trees, support vector machines, regression analyses, Bayesian networks, Gaussian processes, genetic algorithms, and artificial neural networks (or, simply, neural networks). The model structure 620 can include a number of structure layers, a number of nodes (or neurons) at each structure layer, and activation functions of each node. Each node's activation function defines how a node converts data received to data output. The structure layers may include an input layer of nodes that receive input data, an output layer of nodes that produce output data. The model structure 620 may include one or more hidden layers of nodes between the input and output layers. The model structure 620 can be an Artificial Neural Network (or, simply, neural network) that connects the nodes in the structured layers such that the nodes are interconnected. Examples of neural networks include Feedforward Neural Networks, convolutional neural networks (CNNs), Recurrent Neural Networks (RNNs), Autoencoder, and Generative Adversarial Networks (GANs).

The model parameters 622 represent the relationships learned during training and can be used to make predictions and decisions based on input data. The model parameters 622 can weight and bias the nodes and connections of the model structure 620. For instance, when the model structure 620 is a neural network, the model parameters 622 can weight and bias the nodes in each layer of the neural networks, such that the weights determine the strength of the nodes and the biases determine the thresholds for the activation functions of each node. The model parameters 622, in conjunction with the activation functions of the nodes, determine how input data is transformed into desired outputs. The model parameters 622 can be determined and/or altered during training of the algorithm 616.

The loss function engine 624 can determine a loss function, which is a metric used to evaluate the AI model's performance during training. For instance, the loss function engine 624 can measure the difference between a predicted output of the AI model 630 and the actual output of the AI model 630 and is used to guide optimization of the AI model 630 during training to minimize the loss function. The loss function may be presented via the ML framework 614, such that a user can determine whether to retrain or otherwise alter the algorithm 616 if the loss function is over a threshold. In some instances, the algorithm 616 can be retrained automatically if the loss function is greater than the threshold. Examples of loss functions include a binary-cross entropy function, hinge loss function, regression loss function (e.g., mean square error, or quadratic loss), mean absolute error function, smooth mean absolute error function, log-cosh loss function, and quantile loss function.

The optimizer 626 adjusts the model parameters 622 to minimize the loss function during training of the algorithm 616. In other words, the optimizer 626 uses the loss function generated by the loss function engine 624 as a guide to determine what model parameters lead to the most accurate AI model. Examples of optimizers include Gradient Descent (GD), Adaptive Gradient Algorithm (AdaGrad), Adaptive Moment Estimation (Adam), Root Mean Square Propagation (RMSprop), Radial Base Function (RBF) and Limited-memory BFGS (L-BFGS). The type of optimizer 626 used may be determined based on the type of model structure 620 and the size of data and the computing resources available in the data layer 602.

The regularization engine 628 executes regularization operations. Regularization is a technique that prevents over-and under-fitting of the AI model 630. Overfitting occurs when the algorithm 616 is overly complex and too adapted to the training data, which can result in poor performance of the AI model 630. Underfitting occurs when the algorithm 616 is unable to recognize even basic patterns from the training data such that it cannot perform well on training data or on validation data. The optimizer 626 can apply one or more regularization techniques to fit the algorithm 616 to the training data properly, which helps constraint the resulting AI model 630 and improves its ability for generalized application. Examples of regularization techniques include lasso (L1) regularization, ridge (L2) regularization, and elastic (L1 and L2 regularization).

The application layer 608 describes how the AI system 600 is used to solve problem or perform tasks. In an example implementation, the application layer 608 can include software implemented on the controller 124 and/or computer device 168 shown by FIG. 1.

### Industrial Applicability

The disclosed apparatuses and systems have broad industrial applicability across gas turbine engine applications in power generation, oil and gas, and industrial facilities. The lube oil temperature control valve monitoring system can be implemented on both new and existing gas turbine engines to prevent costly unplanned shutdowns and equipment damage. The technology is beneficial for facilities operating in high ambient temperature conditions where proper oil cooling is critical for engine reliability. Implementation can be performed using temperature sensors and software updates to existing control systems, making it cost-effective to deploy across large fleets. The predictive maintenance capabilities enable facilities to improve maintenance scheduling and reduce operational costs by addressing valve and heat exchanger issues before they cause shutdowns. The artificial intelligence (AI) system can be integrated with existing remote monitoring infrastructure to provide automated analysis and alerts. This technology helps industrial facilities maintain reliable operation of critical rotating equipment by ensuring proper lubrication and cooling system performance. The disclosed systems'abilities to differentiate between valve and cooling system issues enable targeted maintenance actions that reduce equipment downtime.

The benefits and advantages of the implementations described herein include early detection of temperature control valve degradation for gas turbine engine operation and maintenance through temperature measurements and AI analysis. The disclosed methods prevent unexpected shutdowns that can cause costly downtime and potential engine damage. The disclosed apparatuses enable maintenance to be scheduled proactively before peak ambient temperature periods when cooling issues are critical. The disclosed systems prevent the need for additional position sensors by calculating valve position from existing temperature measurements, making it cost-effective to implement on both new and existing engines.

Beyond valve monitoring, the disclosed technology can detect heat exchanger performance degradation, fan/belt failures, and cooling system issues by analyzing temperature patterns and heat rejection calculations. The notifications provided include specific maintenance recommendations and timing guidance based on seasonal temperature patterns. The disclosed monitoring approaches maintain preferred oil temperatures for proper bearing lubrication and cooling while reducing maintenance costs through prevention rather than reactive repairs. The technology can be applied across different gas turbine engine platforms and cooling system configurations.

FIG. 7 is a block diagram that illustrates an example of a computer system 700 in which at least some operations described herein can be implemented. Components of the computer system 700 can be used to implement the controller 124 and computer device 168 shown by FIG. 1.

As shown, the computer system 700 can include: one or more processors 702, main memory 706, non-volatile memory 710, a network interface device 712, video display device 718, an input/output device 720, a control device 722 (e.g., keyboard and pointing device), a drive unit 724 that includes a storage medium 726, and a signal generation device 720 that are communicatively connected to a bus 716. The bus 716 represents one or more physical buses and/or point-to-point connections that are connected by appropriate bridges, adapters, or controllers. Various common components (e.g., cache memory) are omitted from FIG. 7 for brevity. Instead, the computer system 700 is intended to illustrate a hardware device on which components illustrated or described relative to the examples of the figures and any other components described in this specification can be implemented.

The computer system 700 can take any suitable physical form. For example, the computer system 700 can share a similar architecture as that of a server computer, personal computer (PC), tablet computer, mobile telephone, game console, music player, wearable electronic device, network-connected (“smart”) device (e.g., a television or home assistant device), AR/VR systems (e.g., head-mounted display), or any electronic device capable of executing a set of instructions that specify action(s) to be taken by the computer system 700. In some implementation, the computer system 700 can be an embedded computer system, a system-on-chip (SOC), a single-board computer system (SBC) or a distributed system such as a mesh of computer systems or include one or more cloud components in one or more networks. Where appropriate, one or more computer systems 700 can perform operations in real-time, near real-time, or in batch mode.

The network interface device 712 enables the computer system 700 to mediate data in a network 714 with an entity that is external to the computer system 700 through any communication protocol supported by the computer system 700 and the external entity. Examples of the network interface device 712 include a network adaptor card, a wireless network interface card, a router, an access point, a wireless router, a switch, a multilayer switch, a protocol converter, a gateway, a bridge, bridge router, a hub, a digital media receiver, and/or a repeater, as well as all wireless elements noted herein.

The memory (e.g., main memory 706, non-volatile memory 710, machine-readable medium 726) can be local, remote, or distributed. Although shown as a single medium, the machine-readable medium 726 can include multiple media (e.g., a centralized/distributed database and/or associated caches and servers) that store one or more sets of instructions 728. The machine-readable (storage) medium 726 can include any medium that is capable of storing, encoding, or carrying a set of instructions for execution by the computer system 700. The machine-readable medium 726 can be non-transitory or include a non-transitory device. In this context, a non-transitory storage medium can include a device that is tangible, meaning that the device has a concrete physical form, although the device can change its physical state. Thus, for example, non-transitory refers to a device remaining tangible despite this change in state.

Although implementations have been described in the context of fully functioning computing devices, the various examples are capable of being distributed as a program product in a variety of forms. Examples of machine-readable storage media, machine-readable media, or computer-readable media include recordable-type media such as volatile and non-volatile memory devices 710, removable flash memory, hard disk drives, optical disks, and transmission-type media such as digital and analog communication links.

In general, the routines executed to implement examples herein can be implemented as part of an operating system or a specific application, component, program, object, module, or sequence of instructions (collectively referred to as “computer programs”). The computer programs typically include one or more instructions (e.g., instructions 704, 708, 728) set at various times in various memory and storage devices in computing device(s). When read and executed by the processor 702, the instruction(s) cause the computer system 700 to perform operations to execute elements involving the various aspects of the disclosure.

We claim:

1. A lube oil cooling system for a gas turbine engine, comprising:
a lube oil tank configured to store lube oil;
a pump configured to circulate the lube oil from the lube oil tank through the lube oil cooling system;
a heat exchanger configured to cool the lube oil;
a temperature control valve having:
a first inlet port configured to receive the lube oil from the lube oil tank;
a second inlet port configured to receive cooled lube oil from the heat exchanger; and
an outlet port configured to provide temperature-controlled lube oil to the gas turbine engine;
one or more temperature sensors configured to measure at least one of a lube oil tank temperature, a heat exchanger outlet temperature, or a header temperature downstream of the outlet port; and
a controller configured to:
receive temperature measurements from the one or more temperature sensors;
determine a position of the temperature control valve based on the temperature measurements;
determine degradation of the temperature control valve by comparing the position of the temperature control valve to an expected position at a given lube oil temperature; and
generate a notification when the degradation of the temperature control valve is detected.

2. The lube oil cooling system of claim 1, wherein the temperature control valve comprises:
a valve housing defining internal passages to direct and mix the lube oil from the first inlet port and the cooled lube oil from the second inlet port.

3. The lube oil cooling system of claim 1, wherein the temperature control valve comprises:
a wax element disposed within a valve housing that expands and contracts with temperature changes; and
a spring mechanism coupled to the wax element to modulate lube oil flow between the first inlet port and the second inlet port.

4. The lube oil cooling system of claim 1, wherein the controller is configured to:
monitor changes in a lube oil temperature at which the temperature control valve begins opening over time;
determine, based on the changes in the lube oil temperature, a rate of change in the lube oil temperature at which the temperature control valve begins opening over time, and
predict when the temperature control valve will fail based on the rate of change.

5. The lube oil cooling system of claim 1, wherein the controller is configured to:
determine a characteristic curve representing expected positions of the temperature control valve across a range of temperature differences between the lube oil tank and a heat exchanger outlet; and
compare actual positions of the temperature control valve to the characteristic curve to detect deviations, wherein the degradation of the temperature control valve is determined when the deviations exceed predetermined thresholds.

6. The lube oil cooling system of claim 1, wherein the controller is configured to:
determine a bypass flow percentage based on the temperature measurements using; and
monitor changes in the bypass flow percentage over time, wherein the degradation of the temperature control valve is determined when the bypass flow percentage deviates from expected values at given operating temperatures.

7. The lube oil cooling system of claim 1, wherein the controller is configured to generate at least one of heat exchanger degradation alerts indicating cleaning requirements, temperature control valve service recommendations, or alerts indicating fan, belt, or coolant pump failures.

8. A gas turbine engine system, comprising:
a gas turbine engine;
a lube oil cooling system comprising:
a lube oil tank configured to store lube oil;
a pump configured to circulate the lube oil from the lube oil tank through the gas turbine engine system;
a heat exchanger configured to cool the lube oil;
a temperature control valve configured to:
receive lube oil from the lube oil tank and cooled lube oil from the heat exchanger; and
provide temperature-controlled lube oil to the gas turbine engine; and
one or more temperature sensors configured to measure at least a lube oil tank temperature; and
a controller configured to:
determine a position of the temperature control valve based on temperature measurements by the one or more temperature sensors; and
determine degradation of the temperature control valve by comparing the position of the temperature control valve to an expected position at a given lube oil temperature.

9. The gas turbine engine system of claim 8, wherein the lube oil cooling system comprises:
pipe fittings configured to mechanically couple a first inlet port of the temperature control valve to the lube oil tank and a second inlet port of the temperature control valve to the heat exchanger.

10. The gas turbine engine system of claim 8, wherein the lube oil cooling system is configured to maintain header temperature below a shutdown threshold by circulating the cooled lube oil through the gas turbine engine.

11. The gas turbine engine system of claim 8, wherein the controller is configured to:
establish baseline temperature control valve operation by monitoring positions of the temperature control valve across operating temperatures; and
predict failure of the temperature control valve based on deviations from the baseline temperature control valve operation.

12. The gas turbine engine system of claim 8, wherein the controller is configured to:
monitor a valve opening temperature at which the temperature control valve begins diverting flow of the lube oil to the heat exchanger; and
detect when the valve opening temperature increases above a predetermined threshold.

13. The gas turbine engine system of claim 8, wherein the controller is configured to:
analyze impact of seasonal temperature variations on operation of the temperature control valve using an artificial intelligence (AI) system; and
generate notifications recommending maintenance of the temperature control valve based on at least one of detected valve degradation, historical seasonal temperature patterns, or predicted failure of the temperature control valve.

14. The gas turbine engine system of claim 8, wherein the controller is configured to determine the degradation of the temperature control valve based on temperature differentials between lube oil header temperature, heat exchanger outlet temperature, and the lube oil tank temperature.

15. A method of detecting degradation of a lube oil temperature control valve, comprising:
receiving, from one or more temperature sensors, temperature measurements of at least one of a lube oil tank temperature, a heat exchanger outlet temperature, or a header temperature downstream of the temperature control valve;
determining, using an artificial intelligence (AI) system, a position of the temperature control valve based on the temperature measurements;
determining degradation of the temperature control valve by comparing the position of the temperature control valve to an expected position at a given lube oil temperature;
predicting failure of the temperature control valve based on the determined degradation; and
generating a notification indicating the predicted failure.

16. The method of claim 15, comprising:
determining a characteristic curve representing expected positions of the temperature control valve across operating temperatures; and
comparing the position of the temperature control valve to the characteristic curve.

17. The method of claim 15, wherein predicting the failure of the temperature control valve comprises at least one of:
monitoring a temperature at which the temperature control valve begins diverting flow of the lube oil to the heat exchanger; or
detecting when an opening temperature of the temperature control valve increases above approximately 135°F.

18. The method of claim 15, comprising:
extracting, by a feature extraction module, a feature vector from temperature data; and
training the AI system based on operational data and detected degradation patterns.

19. The method of claim 15, comprising detecting degradation of the heat exchanger by:
determining a heat rejection value of the heat exchanger based on the position of the temperature control valve;
comparing the heat rejection value to an expected heat rejection model.

20. The method of claim 15, wherein the degradation of the temperature control valve is determined based on temperature differentials between the header temperature, the heat exchanger outlet temperature, and the lube oil tank temperature.
